(19) United States
(12) Patent Application Publication
(10) Pub. No.: US 2012/0157420 A1
(43) Pub. Date: Jun. 21, 2012

(54) TREATMENT AND PREVENTION OF SECONDARY INJURY AFTER TRAUMA OR DAMAGE TO THE CENTRAL NERVOUS SYSTEM

(76) Inventor: Eric B Schneider, Glen Arm, MD (US)

(21) Appl. No.: 13/392,371

(22) PCT Filed: Aug. 31, 2010
(86) PCT No.: PCT/US10/47206

§ 371 Date: Feb. 24, 2012

Related U.S. Application Data
(60) Provisional application No. 61/238,453, filed on Aug. 31, 2009.

(51) Int. Cl. A61K 31/56; A61K 31/405; A61K 31/215; A61K 31/4418; A61K 31/505; A61P 29/00; A61K 31/47; A61P 25/00; A61P 3/06; A61P 9/00; A61P 25/22; A61P 25/24; A61K 31/366; A61K 31/40

(52) U.S. Cl. 514/171; 514/460; 514/419; 514/510; 514/277; 514/275; 514/423; 514/311

(57) ABSTRACT

A method of administering one or multiple medications to human patients with CNS injury through oral or parenteral (including transdermal, intravenous, subcutaneous, intramuscular) routes. Inflammatory and immunological processes have been shown to cause secondary damage to CNS tissues in individuals with acute CNS injury. The present invention administers one or more of the following medications, which have properties that mitigate the inflammatory and immunological processes that lead to secondary CNS damage, via trans-dermal absorption: a statin compound (e.g., a HMG-CoA reductase inhibitor), a progesterone compound, or a cholinesterase inhibiting compound, among others, either alone or in combination with other compounds.

### FIELD

This application describes methods of treatment or prevention of secondary injury to the human central nervous system after trauma or damage (both internal and external in origin) and pharmaceutical preparations for use in such methods, as well as methods of manufacture of such preparations.

### BACKGROUND

Brain injury and dementia are significant public health issues at present and are likely to grow in importance as the population ages. Nearly 1.5 million individuals suffer a traumatic brain injury (TBI) each year and 5.3 million individuals are living with disabilities that were acquired through TBI. Among those who suffer brain trauma in the United States every year, more that 50,000 die as a result of their injury. Further, 235,000 individuals with brain injury require hospitalization and approximately 1.1 million are treated in emergency medical facilities and released. (Brain Injury Association [B.I.A.], 2006)

Acute incidents that damage the central nervous system (CNS) including the brain, such as but not limited to TBI, have been implicated as possible contributors to increased risk of developing chronic neurological and neuropsychiatric illness, including dementia and parkinsonism among others, across the lifespan. Looking at Alzheimer's disease (AD), which is just one of many chronic neuropsychiatric illnesses, it is projected that there will be approximately 8,640,000 individuals in the U.S. alone living with a diagnosis of AD by the middle of this century. The economic, social and family burdens associated with caring for this number of individuals are daunting.

To date, no pharmacological therapy has been available to treat acute trauma to the central nervous system (including the brain and spinal cord) at or very near the time of injury with the aim of preventing secondary damage related to naturally occurring internal inflammatory and immunological responses in the CNS. Acute traumas to the CNS include traumatic brain injury, traumatic spinal cord injury, ischemic and hemorrhagic events (e.g., stroke), transient ischemic attack, encephalopathies (including but not limited to viral encephalitis and acute disseminated encephalomyelitis), cerebral anoxia, and any other event where the CNS is acutely damaged such that inflammatory and immunological processes are activated. A continuing and unmet need exists for new and innovative medical treatments for the prevention and treatment of post-traumatic secondary injury to the CNS.

Statin use has been associated with reduced mortality and reduced damage to brain tissue in controlled trials of animals subjected to traumatic brain injury or induced ischemic stroke (Elkind, et al., 2005). While the underlying mechanisms are not clearly understood, it is suspected that statins may function as “potent vascular anti-inflammatory agents” (Laio, 2004). Better functional outcomes, as measured using spatial learning tasks, have been reported in statin-treated rats subjected to brain infarction/brain trauma than in similarly injured rats not treated with statins (Lu, et al., 2004).

Inflammation in the central nervous system is not limited to acute conditions but is also present in chronic progressive conditions leading to dementia. From the time of Alois Alzheimer, neuro-inflammation has also been implicated as a factor in dementia. At this point, population-based studies are equivocal as to a potential moderating role for statins in late life dementia. It seems likely that the long-term inflammatory processes associated with many chronic neurological and neuropsychiatric illnesses may either commence as a result of neuro-immuno-biochemical process associated with acute CNS insult or that these long term inflammatory processes may be abetted by acute insults and the body's reaction to them.

At present, there are limited pharmacological interventions available to reduce the impact or delay the onset of dementing conditions in either acute or chronic settings. Available agents, including Aricept, Namenda and Memantine, may delay disability or slow deterioration to a small extent, but these agents do not prevent or reverse the course of dementia. It is important to note that many of the studies on the cost effectiveness of these medications were sponsored by the pharmaceutical industry (see: Loveman, et al. 2006). Appropriately applied, the therapeutic interventions for acute CNS injury described herein may play a role in reducing the long-term risk of chronic CNS illness and in lowering the burdens associated with these devastating diseases. WO 2006/002127, WO 2006/119598, WO 2009/080301, WO 2007/106862, WO 2003/086379 are all incorporated herein by reference in their entirety.

### SUMMARY

The present invention provides methods for the treatment and prevention of acute CNS injury whereby secondary damage to the CNS is mitigated or eliminated. The new methods described herein are especially useful in treating patients who are unconscious, intubated or who otherwise cannot accept medications via oral administration. The present invention provides a method of treating or preventing secondary injury in a patient with a brain injury where a statin is administered to a patient. It also provides a method of treating or preventing secondary injury in a patient currently on lipid-lowering therapy by administering a statin to a patient. The invention provides a method of treating or preventing secondary injury in a patient at risk for a brain injury by prophylactically administering a statin to a patient.

In one aspect, the present invention includes a method of administering one or multiple medications to human patients with CNS injury through oral or parenteral (including transdermal, intravenous, subcutaneous, intramuscular) routes. Inflammatory and immunological processes have been shown to cause secondary damage to CNS tissues in individuals with acute CNS injury. It is another aspect of the present invention to administer one or more of statin-like medications, which have properties that mitigate the inflammatory and immunological processes that lead to secondary CNS damage, via trans-dermal absorption: a statin compound (e.g., a HMG-CoA reductase inhibitor), a progesterone compound, or a cholinesterase inhibiting compound, among others, either alone or in combination with other compounds.

In humans, statin use is associated with decreased rates of severe inflammatory reactions, such as sepsis, in individuals with bacterial infections (Almog, et al. 2004). The systemic inflammatory cascade seen in sepsis bears some resemblance to the inflammatory processes seen in patients with traumatic injuries. Statins may have significant anti-inflammatory effects in humans beyond their antihyperlipidemic qualities (see: Liappis, et al., 2001). Statin use may lower the risk of mortality and improve the long-term outcome for individuals who have suffered a traumatic brain injury.

Additional features may be understood by referring to the accompanying drawings, which should be read in conjunction with the following detailed description and examples.

### DESCRIPTION OF THE DRAWINGS

FIG. 1 is a side view of devices for the transdermal administration of CNS protective medications in accordance with an exemplary embodiment hereof

FIG. 2 is a schematic diagram of a mechanism for controlling electrophoretic/iontophoretic transdermal transmission of CNS protective medications.

FIG. 3 is diagram of an exemplary patch with mechanical abrasion devices (e.g., microneedles) to enhance penetration through the stratum corneum for transmission of CNS protective medications.

FIGS. 4-5 are schematic diagrams illustrating exemplary transdermal absorption methods enhanced by ultrasonic transmission of CNS protective medications.

FIG. 6 is schematic diagram illustrating an exemplary transdermal absorption enhanced by thermal ablation techniques to increase permeability of the skin prior to improve transmission of CNS protective medications.

FIG. 7 is a schematic diagram illustrating an exemplary transdermal transmission of CNS protective medications via high pressure liquid jet infusion techniques.

FIG. 8 is a schematic diagram illustrating an exemplary transdermal transmission of CNS protective medications via high pressure solid jet infusion techniques.

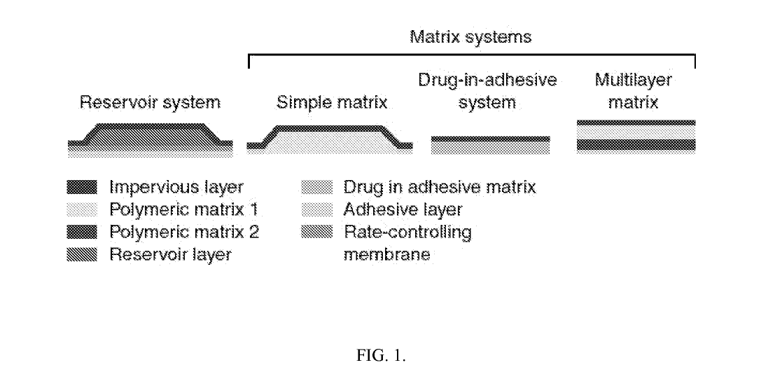

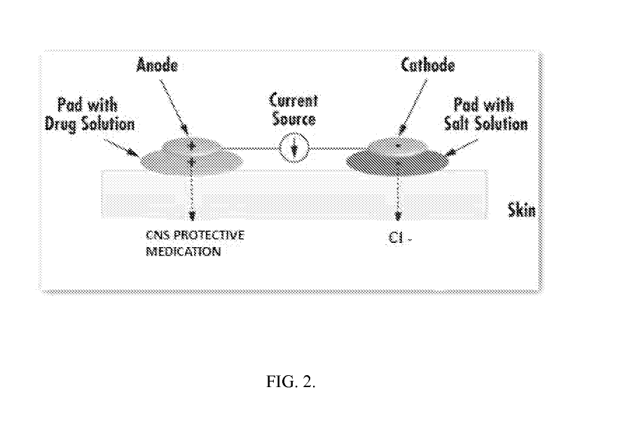

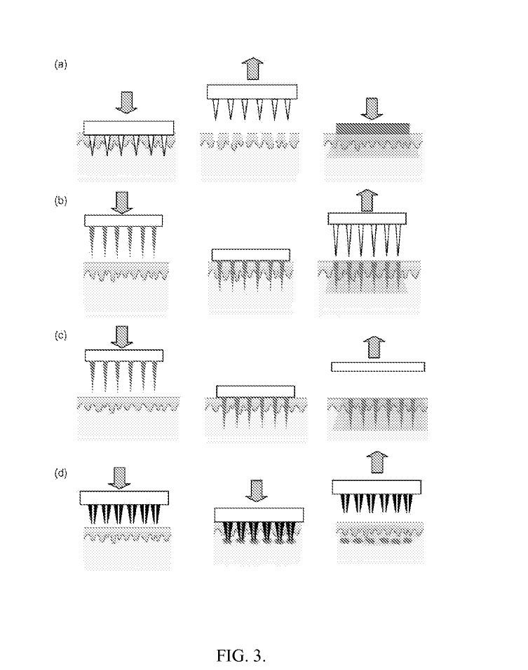

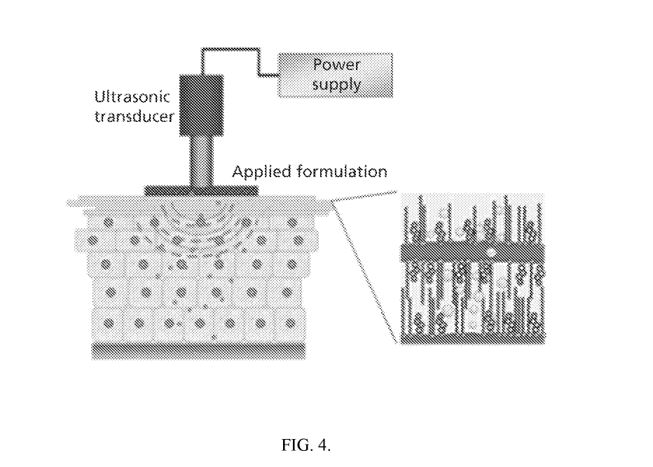

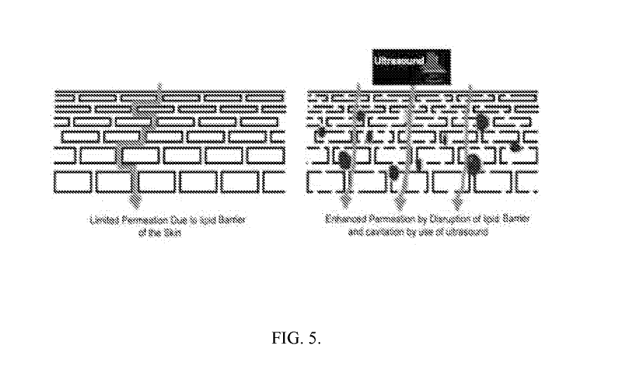

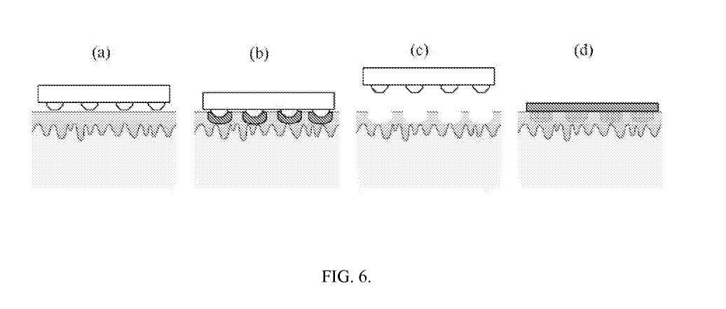

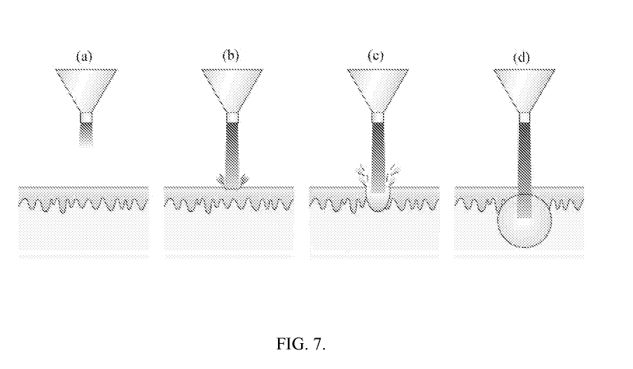

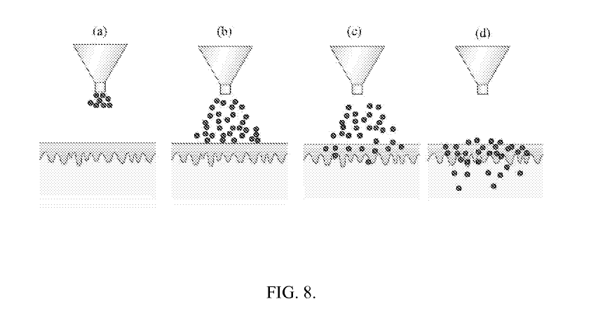

### DETAILED DESCRIPTION

The inventor has discovered that the administration of certain pharmaceutical compounds mitigates the secondary injury resulting from trauma to the brain and more generally to the central nervous system. Accordingly, the present invention provides a method for the treatment or prevention of secondary injury following traumatic injury. The method comprises administering to a patient a statin.

The invention provides a method of treating or preventing secondary injury in a patient with a brain injury where a statin is administered to a patient. The invention also provides for a method of treating or preventing secondary injury in a patient currently on lipid-lowering therapy by administering a statin to a patient. The statin would be a substitute for said lipid-lowering therapy and is administered to a patient after a trauma that causes a brain injury. The invention also provides for a method of treating or preventing secondary injury in a patient at risk for a brain injury where a statin is prophylactically administered to a patient. The invention also provides for the above methods where a patient is not otherwise receiving a statin in a lipid-lowering therapy. The statin can be an HMG-CoA reductase inhibitor selected from the group consisting of lovastatin, simvastatin, fluvastatin, pravastatin, cerivastatin, rosuvastatin, atorvastatin, pitavastatin, and combinations thereof. The statin is administered transdermally and prevents or mitigates endothelial dysfunction in said patient. The invention can be used with a patient that has a history or cardiovascular disease, where the patient could be an athlete, military personnel, an emergency responder, or a human child. The brain injury in the above said circumstances is a traumatic brain injury and the statin mitigates the secondary effects of such brain injury. The statin treats or prevents symptoms selected from the group consisting of transient confusion, loss of consciousness, becoming easily fatigued, disordered sleep, headache, vertigo or dizziness, irritability or aggression, anxiety, depression, personality changes, apathy or lack of spontaneity, amnesia, and other neurologic deficits. The brain injury can also be a mild, moderate, or severe trauma such as a concussion, coma, inflammation, a thromboembolism, or a hemorrhage. The statin can be co-administered with any medicine recited in this disclosure such as a beta-blockage agent, an alpha/beta-androgenic blocking agent, an anti-inflammatory azole compound, a choline esterase inhibitor, a calcium-channel blocker, an NMDA-receptor antagonist, an antihistamine, a steroid, or a COX-2 inhibitor.

In trauma, brain injury is a common cause of death, and complications from closed-head traumatic injuries are a significant cause of morbidity and mortality in patients who reach the hospital alive. Traumatic injury to the brain involves a primary brain injury that occurs at impact and leads to disruption of brain substance and blood vessels among other things. Primary injury to the head may cause disruption of neurons, glial cells, and microvasculature localized at the area of impact. As the brain decelerates within the skull, it is also vulnerable to countercoup injury, which in some cases is the more severe site of damage. Diffuse injury from brain distortion, particularly when the mechanism includes rotational forces, can lead to damage to deep brain structures, which can then lead to widespread disruption of white matter axons throughout the brain. Such injuries often produce damage to a large number of neurologic systems. In addition to the primary injury, secondary brain injury may result from hypoxia, hypotension, pyrexia, increased intracranial pressure, and altered cellular biochemical processes that are often ongoing long after the primary insult.

According to the present invention statins are used to treat brain injury, including traumatic brain injury (“TBI”), and to mitigate the effects of secondary injury. Examples of brain injuries include mild, moderate, and severe traumas (including blunt force trauma and penetrating injuries), concussion, coma, or any other trauma to the head, including internal traumas (e.g., inflammation, thromboembolism, hemorrhage), that result in symptoms consistent with brain injury. Such symptoms may include transient confusion, loss of consciousness, becoming easily fatigued, disordered sleep, headache, vertigo or dizziness, irritability or aggression, anxiety, depression, personality changes, apathy or lack of spontaneity, amnesia, or other neurologic deficits. Statins (HMG-CoA reductase inhibitors) may also be used to treat stroke, intra-cranial hemorrhage (“ICH”), and other acute neuro-inflammatory conditions such encephalitis, ADEM, and meningitis, among others. Moreover, statins may also be used to treat chronic medical conditions, such as Alzheimer's disease, fronto-temporal dementia, multiple sclerosis, transverse myelitis, Parkinson's disease, and progressive supra-nuclear palsy, among others.

The present invention uses a variety of statin medications suitable for CNS protection as disclosed herein, including lovastatin (MEVACOR), simvastatin (ZOCOR), fluvastatin sodium (LESCOL), pravastatin sodium (PRAVACHOL), cerivastatin sodium (BAYCOL), rosuvastatin calcium, atorvastatin calcium (LIPITOR), and pitavastatin, among others. Statin are HMG-CoA reductase inhibitors, which inhibit the conversion of HMG-CoA to mevalonate, an early and rate-limiting step in the biosynthesis of cholesterol in the liver.

In the present invention the site of action may be within the endothelium in the brain. As such, routes of administration that by-pass hepatic first-pass metabolism are preferred, especially transdermal routes, such as those routes illustrated in the attached drawings. Transdermal drug delivery is an attractive route because of the controlled dosage of the statin and avoidance of the hepatic first-pass effect. Moreover, most statins are lipophilic, and therefore they can cross all layers of the skin and enter circulation.

Transdermal delivery also provides a sustained and consistent delivery of medication, avoiding peaks and valleys in blood levels that are often associated with oral dosage forms. Thus, using transdermal delivery, one can administer lower doses of drug to achieve the same therapeutic effect compared to oral administration, reducing or eliminating dose-dependent side effects. The circulating concentration of transdermally administered statins may in fact be higher than that achieved with an equivalent dosage of oral statins because first-pass hepatic metabolism is avoided. Additionally, patients with traumatic brain injury are often intubated, and therefore oral administration is impossible. Because most statins are so lipophilic, it is difficult to formulate them for intravenous administration. For all of the foregoing reasons, transdermal administration of statins is preferred.

For transdermal administration, transdermal patches may be used. Transdermal patches typically include a backing and an adhesive, which adhere to the skin. The statin, along with any excipients, preservatives, permeation enhancers, solvents, or other active or inactive ingredients, is held in contact with the skin. Other components may also be included in the patch, e.g., a membrane, which controls the release of the statin. Such an adhesive device, when affixed to the skin of a human, introduces a statin medication to the bloodstream via the transdermal route. This statin medication acts to limit damage to the CNS by reducing destructive effects of normal inflammatory and immunological processes that occur in the CNS as a result of trauma or insult.

When administered orally, the statin may be formulated as a solid dosage form, e.g., a tablet. Suitable oral dosage forms may include the statin with suitable binding agents, lubricants, preservatives, humectants, wicking agents, or other excipients.

Combination products may include the statin in addition to another active ingredient. For example, the pharmaceutical formulation may include statins in combination with cholinesterase inhibitiors (or other drugs) for administration via oral route. Likewise, statins may be formulated in combination with cholinesterace inhibitiors (or other drugs) for administration via the transdermal route.

Additional compounds suitable for use in combination therapy with statins in the treatment of CNS trauma include the following: reversible acetylcholinesterase inhibitors (galantamine or galanthamine (NIVALIN, RAZADYNE, RAZADYNE ER, REMINYL), physostigmine (also known as eserine), neostigmine, pyridostigmine (MESTINON, REGONOL), ambenonium (MYTELASE), donepezil (ARICEPT), edrophonium (TENSILON, ENLON, REVERSOL), and tacrine (COGNEX)); reversible acetylcholinesterase/butyrylcholinesterase inhibitors (rivastigmine (EXELON)); antihistimines (dimebolin hydrochloride (DIMEBON)); NMDA receptor agonists (memantine (AXURA, AKATINOL, NAMENDA, EBIXA, ABIXA, MEMOX)); and others (alpha-GPC, L-alpha glycerylphosphorylcholine (alpha GPC, choline alfoscerate), lecozotan, nicergoline, semagacestat)). Additionally, cardiovascular drug such as nifedipine (PROCARDIA), verapamil (CALAN) or clopidogrel (PLAVIX) may be combined with statins for CNS protection. Calcium channel blocking medications suitable for use in combination products include amlodipine (NORVASC), diltiazem (CARDIZEM LA, TIAZAC), felodipine (PLENDIL), isradipine (DYNACIRC), nifedipine (ADALAT, PROCARDIA), nicardipine (CARDENE), nimodipine (NIMOTOP), nisoldipine (SULAR), and verapamil (COVERA-HS, VERELAN PM, CALAN). Betablocade agents may also be included in combination products, such as acebutolol hydrochloride (SECTRAL), atenolol (TENORMIN), betaxolol hydrochloride (KERLONE), bisoprolol fumarate (ZEBETA), carteolol hydrochloride (CARTROL), esmolol hydrochloride (BREVIBLOC), metoprolol (LOPRESSOR, TOPROL XL), penbutolol sulfate (LEVATOL), nadolol (CORGARD), nebivolol (BYSTOLIC), pindolol (VISKEN), propranolol (INDERAL, INNOPRAN), timolol maleate (BLOCADREN), and sotalol hydrochloride (BETAPACE). Still other compounds suitable for inclusion in combination products include alpha/beta-adrenergic blocking agents, such as carvedilol (COREG) and labetalol hydrochloride (TRANDATE, NORMODYNE).

ADDITIONAL COMPOUNDS SUITABLE FOR USE IN COMBINATION WITH STATIN THERAPY (Developer: Compound)

University of South Florida: (−)-epigallocatechin-3-gallate; National Institutes of Health and Daewoong Pharmaceutical: (−)-phenserine; Fujimoto Seiyaku Co. Ltd.: (R)-(−)-BPAP; Loma Linda University MedicalCenter: (R)-flurbiprofen; GlaxoSmithKline plc/Neurocrine Biosciences, Inc.: 561679; GlaxoSmithKline plc: 644784; GlaxoSmithKline plc: 742457; GlaxoSmithKline plc: 773812; University of Pennsylvania/Avid Radiopharmaceuticals, Inc.: 123I-IMPY; University of Pittsburgh/Uppsala Imanet AB: 18F-6-OH-BTA-1; University of Pennsylvania/Avid Radiopharmaceuticals, Inc.:18F-AV-1/ZK; University of California Los Angeles: 18F-FDDNP; University of Pennsylvania: 18F-labeled fluoropolyethylene glycol derivatives; Taisho Pharmaceuticals Co Ltd: 20-HETE synthase inhibitors; Eli Lilly & Co: 3,5-DHPG; University of Pennsylvania: 5-HT1A ligands; Merck Sharp & Dohme Ltd: 5-HT 2a antagonists; Sigma-Tau Ind Farm Riunite SpA: 5-HT2/dopamine D3 antagonists; NPS Allelix Corp: 5-HT6 antagonists; GlaxoSmithKline plc: 5-HT 6 receptor antagonists; Neurotech Pharmaceuticals Inc.: 5-(pentafluorobenzyl) aminosalicylic acid; University of Pennsylvania/GE Healthcare: 99mTc-Trodat-1-2-; Abbott Laboratories: A-366833: A-35380; ImaRx Therapeutics, Inc. A-74187; Elan Corp plc/Wyeth: AAB-002; Ferrer Internacional SA: Abaperidone; Centocor Inc.: Abciximab; BTG plc: ABIO-08-01:former BTG-1640; Abbott Laboratories: ABT-089; Abbott Laboratories/NeuroSearch A/S: ABT-107; ACADIA Pharmaceuticals Inc.: AC-184897; Dainippon Pharmaceutical Co Ltd.: AC-3933; ACADIA Pharmaceuticals Inc.: AC-90222; Elan Corp plc/Wyeth: ACC-001; University of California/Scripps Institute/Siemens Medical Solutions Molecular Imaging: Acetylcholinesterase inhibitors; University of Bologna: AChE inhibitors; Universitat Autonoma de Barcelona: AChE inhibitors; Synaptica Ltd.: AChE peptide fragment; ACADIA Pharmaceuticals Inc.: ACP-104; Prescient NeuroPharma Inc.: ACPD analogs; Centre National de la Recherche Scientifique (CNRS): ACUDA; Almirall Prodesfarma SA/Neurocrine Biosciences Inc.: Adenosine A2a receptor antagonists; National Institutes of Health: ADNF-14; Addex Pharmaceuticals SA/Ortho-McNeil Pharmaceutical Inc.: ADX-2; Aegera Therapeutics Inc.: AEG-33783: former AEG-3482 series; Aeolus Pharmaceuticals Inc.: AEOL-10113; Aeolus Pharmaceuticals Inc.: AEOL-10150; ArmaGen Technologies Inc.: AGT-100; ArmaGen Technologies Inc.: AGT-120; ArmaGen Technologies Inc.: AGT-140; ArmaGen Technologies Inc.: AGT-160; AGY Therapeutics: AGY-207; Allon Therapeutics Inc.: AL-108; Allon Therapeutics Inc.: AL-208; Allon Therapeutics Inc.: AL-408; University of Queensland: Alpha-conotoxins; OXIS International Inc.: ALT-2074: former BXT-51072; Harvard University: Altropane; VLP Biotech Inc.: Alzheimers disease vaccine; ArQule Inc./Wyeth: Alzheimers therapy; Pharmexa A/S/Lundbeck: Alzheimers vaccine (AutoVac); Johnson & Johnson: Aminopyrimidines; Pfizer Inc.: Ampa antagonists: former PD-159265; Servier: AMPA antagonists: former S-176251:S-34730-1:S-34730; Eli Lilly & Co.: AMPA modulators; NV Organon: AMPA modulators; Yamanouchi Pharmaceutical Co Ltd.: AMPA receptor antagonists; University of Messina/University of Cantanzaro/Pfizer: AMPA receptor antagonists; Novartis AG: AMPA/NMDA modulators: former Methylphenylethynylpyridine (MPEP); Eisai Co Ltd.: AMPA receptor antagonists; Boehringer Ingelheim Corp.: Amyloid-beta formation inhibitors; Mayo Foundation: Amyloid beta MRI contrast agents; Fournier Pharma: Anatibant; Anavex Life Sciences Corp.: ANAVEX-1-41; Neurobiological Technologies Inc.: Ancrod; Acumen Pharmaceuticals Inc. Anti-ADDL antibodies; Aegera Therapeutics Inc.: Anti-apoptotic gene therapy (IAP); Northwestern University/NeuroMedix Inc. Antineuroinflammatories; Isis Pharmaceuticals Inc.: Antisense oligonucleotides (SOD1): former ALS antisense therapeutics ; Antoxis Ltd.: AO-1-530; AcurePharma AB: AP-267; Apogenix Biotechnology AG: APG-101; Bristol-Myers Squibb Co./Pfizer Inc.: Apixaban; Britannia Pharmaceuticals Ltd.: Apomorphine; Arena Pharmaceuticals Inc.: AR-139525; CytRx Corp.: Arimoclomol; Ono Pharmaceutical Co. Ltd.: Arundic acid (injectable formulation): former ONO-2506; Unknown Phase: Axonyx Inc.: AS-602704; Merck Serono SA: AS-602801:former AS-600292: AS-004509: AS-601245: AS-602183; NV Organon: Asenapine; CoMentis Inc.: ATG-Z1; Novartis AG: ATI-355: former 7B12; ARYx Therapeutics Inc.: ATI-5923; Genzyme Corp.: AV-201; Avigen Inc.: AV-333; Aventis SA: AVE-1625; Aventis SA: AVE-8112; Children's Hospital of Boston/Alseras Pharmaceuticals: Axogenesis Factor 1; Acorda Therapeutics Inc.: Axonal guidance proteins; Transition Therapeutics Inc./Elan Corp plc.: AZD-103; AstraZeneca plc: AZD-1080; AstraZeneca plc: AZD-3102; NPS Pharmaceuticals Inc./AstraZeneca plc: AZD-9272; BioAxone Therapeutique: Inc.: BA-1016; Wyeth: BACE 1 inhibitors; Schering-Plough Corp.: BACE 1; Kyoto University: BACE-1 inhibitors; GlaxoSmithKline plc: BACE1 inhibitors-5-; Bristol-Myers Squibb Co.: BACE-1 inhibitors; Johnson & Johnson: BACE inhibitor; The Genetics Company Inc.: BACE inhibitors; Astex Therapeutics Ltd./AstraZeneca plc: BACE inhibitors; Eli Lilly & Co.: BACE inhibitors; Elan Corp plc/Wyeth: Bapineuzumab: former AAB-001; Universita degli Studi di Siena: Benzodiazepine receptor ligands: former PK-11195 analogs; Purdue Neuroscience Corp.: Besonprodil; University of Wisconsin-Madison: Beta amyloid inhibitors; Q-RNA Inc.: Beta-amyloid inhibitors; Oceanix Corp. Beta amyloid inhibitors; Chiesi Farmaceutici SpA: Beta amyloid inhibitors; Sankyo Co Ltd: Beta-amyloid synthesis inhibitors; Merck & Co. Inc. Beta-secretase inhibitors; Actelion: Beta-secretase inhibitors; Sankyo Co. Ltd. BGC-20-1178; Sankyo Co. Ltd./BTG plc: BGC-20-1259; Mitsubishi-Tokyo Pharmaceuticals Inc./Sosei Co. Ltd.: Bifemelane; Preregistration (Phase 3): Solvay SA: Bifeprunox; Vernalis Group plc/Biogen Idec Inc.: BIIB-014: former V-2006; Boehringer Ingelheim Corp.: BIII-890-CL; TorreyPines Therapeutics: Bisnorcymserine; 4SC AG: BK channel blockers; Preregistration: Dainippon Pharmaceutical Co. Ltd.: Blonanserin; Aastrom Biosciences Inc. Bone marrow-derived stem cell therapy; Millennium Pharmaceuticals Inc.: Bortezomib; UCB Pharma SA: Brivaracetam; Blanchette Rockefeller Neurosciences Institute (BNRI)/Aphios Corp.: Bryostatin-1; Knoll Ltd., BTS-72664-6-; Nexia Biotechnologies Inc./PharmaAthene Inc. Butyrylcholinesterase (recombinant human, nerve agent exposure); BioCryst Pharmaceuticals Inc. C1s inhibitors; Cytos Biotechnology AG/Novartis AG: CAD-106; AstraZeneca plc: Calcium channel antagonists; Eli Lilly & Co.: Calcium channel antagonists N-type; Neuropharma SA: Calcium channel blockers; Neuromed Pharmaceuticals Inc.: Calcium channel blockers N-type; Senju Pharmaceutical Co. Ltd.: Calpain inhibitors; H Lundbeck A/S: Carbamylated erythropoietin; Chemical Diversity Research Institute: Caspase-3 inhibitors; Suspended Sunesis Pharmaceuticals Inc.: Caspase inhibitors; Pfizer/Idun Pharmaceuticals Inc.: Caspase inhibitors; Aeolus Pharmaceuticals Inc.: Catalytic antioxidants; University of Toledo: CDD-0102 Alzheimers disease Muscarinic M1 agonist; Cognitive Pharmaceuticals Ltd.: CDD-0304; Pfizer Inc.: Celecoxib; Titan Pharmaceuticals Inc./Bayer Schering: Cell therapy (dopamine producers); National Institutes of Health: Cell transplant therapy; Cephalon Inc.: CEP-26401; Cephalon Inc.: CEP-4143; Ceregene Inc.: CERE-110; Ceregene Inc:. CERE-120; Ceregene Inc.: CERE-130; INSERM: Ciproxifan; Chronogen Inc.: clk-1:former isp-1; Cenomed Pharmaceuticals Inc./Medical College of Georgia: CM-2433; Cogent Neuroscience Inc.: CNIC-568; Cita Neuro-Pharmaceuticals Inc.: CNP-1061:former GT-1061; CeNeS Pharmaceuticals Inc./Ergomed Group: CNS-5161; Clinical ReGen Therapeutics plc: Colostrinin; Pfizer Inc. CP-465022; CSL Behring: CSL-111; Creabilis Therapeutics SpA/Nautilus Biotech SA: CT-500; Cortex Pharmaceuticals Inc./NV Organon/Servier: CX-516; University of California/Cortex/NV Organon/Servier: CX-717: former AMPAKINES; Maas Bio1AB LLC: Cyclosporin A; Glytech Inc./Massachusetts General Hospital: D-serine; Memory Pharmaceuticals Corp.: D-serine reuptake inhibitors; DarPharma Inc.: DAR-201; Alseres Pharmaceuticals Inc.: DAT inhibitors: former BLS-602: BLS-605; Development Centre for Biotechnology: DCB-AD1; DiverDrugs SL: DD-20207; Debiopharm SA: DEBIO-9902 (sc, sustained release implant): former ZT-1 (injectable, sustained release); Memorial Sloan-Kettering Cancer Center/Progenics Pharmaceuticals Inc.: Dehydroascorbic acid (DHA); Seoul University: Dehydroevodiamine; Toray Industries Inc.: Delta opioid antagonists: former Naltrindole derivatives; Phase 1:NPS Pharmaceuticals Inc.: Delucemine; Bayer Schering AG/Forest:Laboratories Inc./H Lundbeck A/S: Desmoteplase; Hebrew University of Jerusalem/Pharmos Corp.: Dexanabinol; Preregistration: Irysis Research and Development/AVANIR Pharmaceuticals: Dextromethorphan+quinidine sulfate; Purdue Research Foundation: Dihydrexidine; Bayer Schering Pharma AG: Dihydroquinolines; Medivation Inc.: Dimebon; (Phase 3): Renovis Inc./AstraZeneca plc: Disufenton; Novasite Pharmaceuticals Inc.: Dopamine D1 receptor agonists; GlaxoSmithKline plc 598809: former Dopamine D3 antagonists; Memory Pharmaceuticals Corp.: Dopamine D5 receptor modulators; Purdue University: Doxanthrine; D-Pharm Ltd.: DP-109; D-Pharm Ltd.: DP-460; D-Pharm Ltd.: DP-b99; D-Pharm Ltd.: DP-VPA; Meiji Seika Kaisha Ltd.: DR-2313; Korea Research Institute of Bioscience and Biotechnology: Dykellic acid; Eisai Co. Ltd.: E-2007; Eisai Co. Ltd.: E-2012; Neurocrine Biosciences Inc./Wyeth Research: EAAT-2 inhibitors; Wyeth Research: EAB-318; NsGene A/S: ECT-AD; NsGene A/S: ECT-PD; Universidad Complutense de Madrid: EF-7412; Dr Willmar Schwabe GmbH & Co: EGb-761; Phase 1:ExonHIt Therapeutics SA: EHT-202; Nippon Shinyaku Co. Ltd./PAION AG: Enecadin; University of Tennessee Memphis: EP-475; Mochida Pharmaceutical Co. Ltd.: Epadel: former MND-21; Wyeth Research: E-selectin inhibitors; Novavax Inc.: E-selectin tolerogen (stroke): former Vaccine (stroke); MIGENIX: Estrogen analogs ; ACADIA Pharmaceuticals Inc.: Estrogen receptor beta agonists; GlaxoSmithKline Inc.: Estrogen receptor beta modulators (triazine); Roche Holding AG: EVT-101; Roche Holding AG: EVT-201; Roche Holding AG: EVT-301; Roche Holding AG: EVT-302; ProteoTech Inc.: Exebryl; Pierre Fabre SA: F-14413; Tokyo Metropolitan Institute: F-2-CCG-I; Cortex Pharmaceuticals Inc./NV Organon: Farampator: former Org-24448; Enkam Pharmaceuticals A/S: FGLL; Juvantia Pharma Ltd.: Fipamezole; Astellas Pharma Inc.: FK-1706; Kosan Biosciences Inc.: FK-520 analogs; Fujisawa Pharmaceutical Co. Ltd.: FK-962; Pfizer Inc.: FKBP inhibitors; Harvard University: Fluoratec; Astellas: FR-210575; Merck Sharp & Dohme Ltd.: GABA-A alpha-2/alpha-3 agonists (anxiolytics); University of Wisconsin-Milwaukee/Merck Sharp & Dohme Ltd./Eli Lilly and Co.: GABA-A antagonists; Merck Sharp & Dohme Ltd.: BA modulators; Phase 3 XenoPort Inc.: Gabapentin enacarbil: former XP-13512; Synaptica Ltd/Sanochemia Pharmazeutika AG: Galantamine derivatives; Ceregene Inc: GDNF gene therapy (PD/HD); Centelion SAS/CNRS: Gene therapy (GDNF); Centre Hospitalier/Universitaire Vaudois/University of Zurich: Gene therapy (ALS); Oxford BioMedica plc: Gene therapy (Parkinsons disease); PN Gerolymatos SA: Gero-46; CeNeS Pharmaceuticals Inc.: Acorda Therapeutics Inc.: GGF-2; MGI Pharma Inc.: Glutamate carboxypeptidase II inhibitors: former NAALADase inhibitors; Glaxo Wellcome SpA: Glycine antagonists; Eli Lilly & Co. Glycine transporter inhibitors; Novartis: Glycogen synthase kinase-3 inhibitors; Yuyu Inc./CrystalGenomics Inc.: Glycogen synthase kinase-3beta inhibitors; Amphora Discovery Corp: Glycogen synthase kinase-3beta inhibitors; NPS Allelix/Janssen Pharmaceutica NV: G1yT-1 inhibitors; Pfizer: GMC-1111; MGI Pharma Inc./Symphony Neuro Development Co.: GPI-1485; Wyeth: GSI-953; GlaxoSmithKline plc: GSK-189254A; GlaxoSmithKline plc: GSK-3 inhibitors; AstraZeneca AB: GSK-3 inhibitors; Phase 1:GlaxoSmithKline plc: GSK-933776A; CoMentis Inc.: GTS-21; Bioprojet, Societe Civile de Recherch/Berlin Free University/University College London/INSERM: H3 antagonists; Schering-Plough Research Institute: H3 antagonists; GlaxoSmithKline plc: H3 antagonists; Hunter-Fleming Ltd: HF-0220; Loyola University of Chicago/Hunter-Fleming Ltd.: HF-0420; Curis Inc./Wyeth Pharmaceuticals: Hh agonists; (Discovery):Kyorin Pharmaceutical Co. Ltd: Himbacine analogs; Abbott laboratories: Histamine H3 antagonists; Eli Lilly & Co: Histamine H3 antagonists; MethylGene Inc./EnVivo Pharmaceuticals Inc.: Histone deacetylase inhibitors; Sanofi-Aventis: HP-184; Roche AG: HuCAL anti-beta amyloid monoclonal antibodies; Lay Line Genomics SpA: huIM-13; Keio University/Neurologix Inc.: Humanin gene therapy (Alzheimers); Probiodrug AG: Human glutaminyl cyclase inhibitors; StemCells Inc.: Human neural stem cell therapy; Institute Henri Beaufour: Huntingtons disease therapy: Huntingtons chorea Cyclooxygenase 2 inhibitor: Sodium channel; blocker: Cyclooxygenase 1 inhibitor; Georgetown University/Neuro Hi-Tech/Mayo Foundation: Huperzine A; Neuro-Hitech/Xel Herbaceuticals: Huperzine A; Novartis: Huperzine A analogs; Shanghai Institute of Materia Medica: Huperzine A analogs; Avigen Inc.: Ibudilast: former AV-411; Prescient NeuroPharma Inc.: IGT-440103; Vanda Pharmaceuticals Inc./Novartis AG/Titan Pharmaceuticals Inc.: Iloperidone; Janssen Farmaceutica SA (Spain): Imidazole derivatives; Servier: Imidazolyl nitrones; AlphaRx Inc.: Immunomodulators: former ARX-2000: -2001:-2002; Chiesi Farmaceutici SpA: Indantadol: former CHF-3381; Vernalis plc/Chiesi Farmaceutici SpA: Indantadol: former GT-3381; Pharmadigm INFLABLOC: Androgen receptor agonist; Inotek Pharmaceuticals Corp.: INO-1001; Childrens hospital of Boston/Alseres Pharmaceuticals: Inosine; Nippon Chemiphar Co. Ltd.: Ipenoxazone; NPS Pharmaceuticals Inc.: Isovaleramide: former NPS-1776; Targacept Inc.: Ispronicline: former TC-1734; Preregistration (Phase 3): Kyowa Hakko Kogyo Co. Ltd.: Istradefylline; Celgene Corp.: JNK inhibitors; AstraZeneca plc: JNK-3 inhibitors; Wyeth Research: KChIP/Kv4.3 modulators; H Lundbeck A/S: KCNQ2 potassium channel modulators; Bayer AG: KN-38-7271:former BAY-38-7271; Kyorin Pharmaceutical Co. Ltd. KRP-199; Keryx Biopharmaceuticals Inc.: KRX-411; Merck & Co. Inc.: L-830982; Preregistration (Phase 3): Harris FRC Corp./Schwarz Pharma AG: Lacosamide; Yamanouchi Pharmaceutical Co. Ltd.: Lactacystin; Hebrew University of Jerusalem/Teva Pharmaceutical Industries Ltd.: Ladostigil; Preregistration (Phase 3): GlaxoSmithKline plc: Lamotrigine (extended release): former Lamictal XR; Louisiana University: LAU-0501; Louisiana State University: LAU-8080; Scotia Holdings plc: LAX-101; Wyeth: Lecozotan: former SRA-333; Renovis Inc.: Leukocyte trafficking inhibitors; Voyager Pharmaceutical Corp./DURECT Corp.: Leuprolide acetate implant; Advanced Plant Pharmaceuticals Inc.: LHM-123; Fidia-Georgetown Institute for Neurosciences: LIGA-20; OXIS International Inc.: Lipid soluble antioxidants; Dainippon Sumitomo Pharma Co. Ltd.: Lurasidone: former SM-13496; Lexicon Pharmaceuticals Inc.: LX-6171; University of Chicago/Anagen Therapeutics: LXR agonists; Eli Lilly & Co.: LY-354740; Eli Lilly & Co.: LY-450139; Eli Lilly & Co.: LY-451395; Eli Lilly & Co.: LY-483518; Technion Research & Development Foundation Ltd./Weizmann Institute of Science: M-30; MetaPhore Pharmaceuticals Inc.: M-40401; Medicure Inc.: MC-1; Medicure Inc.: MC-45228; Medicure Inc: MC-45308; Medicure Inc.: MC-5422; Mitsubishi-Tokyo Pharmaceuticals Inc.: MCC-257; Arachnova Ltd.: MCI-225; Bayer AG/Memory Pharmaceuticals Corp.: MEM-1003; Memory Pharmaceuticals Corp.: MEM-1414; Memory Pharmaceuticals Corp.: MEM-1917; Memory Pharmaceuticals Corp.: MEM-3454: former Alpha-7 partial agonists; Memory Pharmaceuticals Corp.: MEM-63908; NeuroMolecular Pharmaceuticals Inc.: Memantine derivatives; Universita di Bologna/Lay Line Genomics SpA: Memoquin; Prescient NeuroPharma Inc.: Mesencephalic astrocyte-derived neurotrophic factor; Eli Lilly & Co.: Metabotropic glutamate receptor agonists; Taisho Pharmaceutical Co. Ltd./Merck & Co. Inc.: Metabotropic glutamate receptor ligands; Pharmacyclics Inc.: Metallotexaphyrins; F Hoffmann-La Roche Ltd., mGluR1 modulator; Merck Research Laboratories/Seaside Therapeutics: mGluR5 antagonists; Addex Pharmaceuticals SA: mGluR5 positive modulators: former ADX-4; Prescient NeuroPharma Inc.: mGLuR agonists; Royal Danish School of Pharmacy/NeuroScience: mGLuR agonists; Mera Pharmaceuticals Inc./Albany Molecular Research: Microalgal-derived compound; Phase 3 Corcept Therapeutics Inc.: Mifepristone; Molecular Insight Pharmaceuticals Inc.: MIP-170D; University of Otago/Antipodean Pharmaceuticals Inc.: Mitoquinone/mitoquinol redox mixture: former MitoQ; Merck & Co. Inc.: MK-0249; Merck & Co. Inc.: MK-0752: former c-7617; Merck & Co. Inc.: MK-0952; Merck & Co. Inc.: MK-677: former c-9136; Mitsubishi-Tokyo Pharmaceuticals Inc. MKC-231; Cephalon Inc./Kyowahakko Kogyo Co. Ltd.: MLK inhibitors; Sapporo Medical University: Modified mesenchymal stem cell therapy; Biopharm GmbH: MP52; ImaRx Therapeutics Inc.: MRX-820; Santen Pharmaceutical Co. Ltd.: Msc-1; ACADIA Pharmaceuticals Inc.: Muscarinic M1 agonists; Eli Lilly & Co.: Muscarinic M1 agonists; Northwestern University: MW01-2-151WH; EpiCept: MX-1013; Phase 1:MIGENIX: MX-4509; MIGENIX: MX-4565: former MITO-4565; Dainippon Sumitomo Pharma Co. Ltd.: Na+/H+ exchange inhibitors; National Institutes of Health: NAPVSIPQ; Neurocrine Biosciences Inc.: NBI-30702; NicOx SA: NCX-2216; Taisho Pharmaceutical Co. Ltd.: NE-100; BIAL Group: Nebicapone: former BIA-3-202; Osaka Bioscience Institute: NEPP-10; Merz & Co. GmbH/Forest Laboratories Inc.: Neramexane; Neurotech Pharmaceuticals Inc./Choongwae Pharma Corp.: Neu-2000; NsGene A/S Biogen Idec Inc.: Neublastin; Clinical: Celmed USA: Neural stem cell therapy; Harvard University/Acorda Therapeutics Inc.: Neuregulin-2; University of Minnesota/MGI GP Inc.: Neuroimmunophilin ligands; Sanofi-Aventis: Neurolysin inhibitors; Abbott Laboratories: Neuronal nAChR ligands; Acorda Therapeutics Inc.: Neuronal stem cell therapy; Vertex Pharmaceuticals Inc./Schering AG: Neurophilins; Renovis Inc: Neuroprotectants (nitrone-based); Medipost Co. Ltd.: Neurostem; Krenitsky Pharmaceuticals Inc.: Neurotrophic factor mimetics; BioVex Ltd.: NeuroVEX; Resverlogix Corp.: NexVas AD; Resverlogix Corp.: NexVas PR; Prana Biotechnology Ltd.: NG-1; Lay Line Genomics SpA: NGF therapy; Life Science Research Israel/TorreyPines Therapeutics Inc.: NGX-267; Neurohealing Pharmaceuticals Inc.: NH-02D; Bayer AG/EnVivo Pharmaceuticals Inc.: Nicotinic acetylcholine receptor agonist; Targacept Inc./Sanofi-Aventis: Nicotinic Ach agonists: former TC-4959; Pfizer Inc.: Nicotinic AChR agonists; Northwestern University: Nitric oxide synthase inhibitors; Pfizer Inc.: Nitric oxide synthase inhibitors; University of Tennessee Memphis/University of Pittsburgh: NMDA antagonists; Pfizer Inc.: NMDA/glycine antagonists; Hoffmann-La Roche AG: NMDA NR2B antagonists (intravenous); University of Maryland/VistaGen Therapeutics Inc.: NMDA receptor antagonists: former 4-Clkynurenine; Merck Sharp & Dohme Research Laboratories: NMDA receptor antagonists (NR2B subtype-selective); Neuromed Pharmaceuticals Inc.: NMED-160 Pain N-type calcium channel blocker; Neuren Pharmaceuticals Ltd.: NNZ-2566 (intravenous, brain injury/stroke); Yale University/Biogen/IDEC Inc.: Nogo receptor modulators; Organix Inc./Harvard Medical School/Massachusetts General Hospital: Nonamines; Bioprojet Pharma/Berlin Free University: Non-imidazole H3 antagonists; Corcept Therapeutics Inc./Argenta Discovery Ltd.: Non-steroidal GR antagonists; Yissum Research Development Co of the Hebrew University of Jerusalem: Nootropic agent; Medinox Inc.: NOX-700; Neuropharma SA: NP-0361; NPS Pharmaceuticals Inc.: NPS-1407; NPS Pharmaceuticals Inc.: NPS-P156; Pfizer Inc.: NR1a/2B subtypeselective NMDA antagonists; NeuroSearch A/S: NS-1209; Mayo Foundation: NT-69-L; Stem Cell Therapeutics Corp.: NTx-265; Nymox Pharmaceutical Corp.: NXD-5150; Nymox Pharmaceutical Corp.: NXD-9062; Janssen Pharmaceutica NV: Ocaperidone; GENOPIA Biomedical GmbH: ODS-II; Organon Laboratories Ltd.: Org-26041; NV Organon: Org-50189; Synthetic Blood International Inc.: Oxycyte; National Institutes of Health: p53 inhibitors; Johnson & Johnson: Paliperidone; Sanofi-Aventis: Paliroden: former SR-57667; Lifegroup SpA: Palmidrol; Panacea Pharmaceuticals Inc.: PAN-811; Aurigene Discovery Technologies Ltd. Pan-caspase inhibitors; Solvay SA: Pardoprunox: former SLV-308; Panacea Pharmaceuticals Inc.: Parkinsons therapeutic peptides (PAN-408, PAN-527); University of Manchester: Parkinsons disease Dopamine receptor agonist: 5-HAT 2c antagonist; Astellas Pharma Inc.: PARP-1 inhibitors; University of Florence/GlaxoSmithKline: PARP-1 inhibitors; Astellas Pharma Inc.: PARP-2 inhibitors; Kyorin Pharmaceutical Co. Ltd.: PARP inhibitors; Octamer Inc./Crimson Pharmaceutical: PARP inhibitors; Wyeth: PAZ-417; Prana Biotechnology Ltd.: PBT-2; Parke-Davis & Co.: PD-148903; Memory Pharmaceuticals Corp/Amgen Inc.: PDE 10 inhibitors; Eli Lilly & Co.: PDE3 inhibitors: former PDE modulators; Memory Pharmaceuticals Corp./Roche Holding AG: PDE 4 inhibitors; University of Auckland/Cornell University: PD gene therapy; CoDa Therapeutics (NZ) Ltd.: Peptagon; Vasogen Inc.: Personalized immunotherapy; Wyeth Research: Perzinfotel; Alexion Pharmaceuticals Inc./The Procter & Gamble Co.: Pexelizumab; TransTech Pharma Inc./Pfizer Inc.: PF-4494700; Clinical: Pfizer Inc.: PH-399733; Sanguine Corp.: PHER-O2; University of Nottingham/H Lundbeck A/S/Royal Danish School of Pharmacy: Philanthotoxins; AGY Therapeutics Inc.: Phosphatase inhibitors; Phase 3 ACADIA Pharmaceuticals Inc.: Pimavanserin tartrate: former ACP-103; PharmaKodex Ltd.: PKX-700; PharmaKodex Ltd.: PKX-963; Thromb-X NV/Geymonat SpA: Placental growth factor; Proneuron Biotechnologies Inc.: PN-277; Wellstat Therapeutics Corp.: PN-401; Pfizer Inc.: PNU-170413; Pfizer Inc.: PNU-177864; Clinical: Polifarma SpA: POL-255; University of Kuopio/Finncovery Oy: POP inhibitor; TorreyPines Therapeutics Inc./NIH Posiphen: former Phenserine; Wyeth Research: Potassium channel modulators; Bristol-Myers Squibb Pharmaceutical Research Institute: Potassium (maxi-K) channel openers; Trigen Holdings AG: PR-15; Samaritan Pharmaceuticals Inc.: Procaine hydrochloride; Vyrex Corp.: Propofol phosphate; Advanced Life Sciences Inc.: Protein aggregation inhibitors; Hebrew University of Jerusalem/Pharmos Corp.: PRS-211375; EPIX Pharmaceuticals Inc.: PRX-03140; EPIX Pharmaceuticals, Inc.: PRX-07034; Proximagen Neuroscience plc: PRX-1; Pharmacopeia Drug Discovery Inc.: PS-246518; Phytopharm plc: PYM-50018; Phytopharm plc: PYM-50028; Quigley Pharma Inc.: QR-333; Roche Holding AG: R-1204; Roche Holding AG: R-1450; Roche Holding AG: R-1497; Roche Holding AG: R-1577; Roche Holding AG: R-1603; Newron Pharmaceuticals SpA: Ralfinamide; Biotica Technology Ltd.: Rapamycin analogs; CardioVascular BioTherapeutics Inc.: Recombinant FGF-1 (injectable, vascular disease); Osaka University/Kringle Pharma Inc./Tripep AB: Recombinant HGF; Thromb-X NV: Recombinant human microplasmin (injected); Galileo Pharmaceuticals Inc.: Redox metabolic modulators (stroke); ReNeuron (UK) Ltd.: ReN-001; ReNeuron (UK) Ltd.: ReN-004; ReNeuron (UK) Ltd.: ReN-005: former Neural stem cell therapy; University of Bristol/ReNeuron (UK) Ltd.: ReN-1820; Phase 3 Valeant Pharmaceuticals: Retigabine: former GKE-841; RepliGen Corp.: RG-1068; Bayer AG/Ortho-McNeil Pharmaceutical Inc.: Rivaroxaban; Sirna Therapeutics Inc./Targeted Genetics Corp.: RNAi therapy; Phase 3 GlaxoSmithKline plc: Rosiglitazone XR; Aderis Pharmaceuticals Inc.: Rotigotine ; Roche Bioscience: RS-100642; Reata Pharmaceuticals Inc.: RTA-404; Shionogi & Co. Ltd/GlaxoSmithKline plc: S-0139; Servier: S-14297; Servier: S-18986; Servier: S-33113-1; Servier: S-33138; Minster Research Ltd.: Sabcomeline; Newron Pharmaceuticals SpA/Merck Serono SA: Safinamide; Wyeth: SAM-315: former 5-HT6 antagonists; Wyeth: SAM-531; Sanofi-Aventis: SAR-502250; Merck KGaA: Sarizotan; Shionogi-GlaxoSmithKline Pharmaceuticals LLC: SB-234551; Discovery (Discontinued): GlaxoSmithKline plc: SB-277011; Schering-Plough Corp.: SCH-420814: former Adenosine A2a antagonists; Scios Inc.: SCIO-323; Scios Inc.: SCIO-469; Taisho Pharmaceutical Co. Ltd.: SEA-0400; Faust Pharmaceuticals: Selective mGluR agonists; UCB SA: Seletracetam; Russian Academy of Sciences: SEMAX; Guilford Pharmaceuticals Inc.: Serine racemase inhibitors; Eisai Co Ltd: Serofendic acids; BioFocus DP: Serotonin antagonists; Eli Lilly & Co./Saegis Pharmaceuticals Inc.: SGS-518; Novartis AG/Saegis Pharmaceuticals Inc.: SGS-742; Schering-Plough Corp.: Siclofen; GlaxoSmithKline plc: SKF-82958; SK Chemicals Life Science: SK-PC-B70M; Solvay SA: SLV-314; Oxford Biomedica plc: SOD1-targeting RNAi therapy (ALS); Merck & Co. Inc.: Somatostatin receptor 2 agonists; Georgetown University/Samaritan Pharmaceuticals Inc.: SP-08; Georgetown University/Samaritan Pharmaceuticals Inc.: SP-233; Sanochemia Pharmazeutika AG: SPH-1371; Biofrontera Bioscience GmbH: Sphingomyelinase inhibitors; Sagami Chemical Research Center/Taisho Pharmaceutical Co. Ltd. Sphingomyelinase inhibitors; Satori Pharmaceuticals Inc.: SPI-014; Wyeth: SRA-444; SIRENADE Pharmaceuticals AG: SRN-003-556; Discovery Sanofi-Aventis: SSR-103800: former G1yT-1 inhibitor; Sanofi-Aventis: SSR-180575; Sanofi-Aventis: SSR-180711; BresaGen Ltd.: Stem cell therapy; Neuralstem Inc.: Stem cell therapy; Samaritan Pharmaceuticals Inc.: Stem cell therapy drugs; Tanabe Seiyaku Co. Ltd/Cell Therapeutics Scandinavia AB/Jichi Medical School: Stem cell therapy; Geron Corp.: Stem cell therapy; University of Miami: Stilbazulenyl nitrone; Daiichi Suntory Biomedical Research Co. Ltd./Taisho Pharmaceutical Co. Ltd.: SUN-N8075; PoliChem SA/SIRENADE Pharmaceuticals AG: Sustained release dihydroergocryptine; Aida Pharmaceuticals Inc.: SY-02; Roche Holding AG/Synosia Therapeutics: SYN-114; Roche Holding AG/Synosia Therapeutics: SYN-115; Roche Holding AG/Synosia Therapeutics: SYN-116; Roche Holding AG/Synosia Therapeutics: SYN-119; Taro Pharmaceutical Industries Ltd.: T-2000; Taro Pharmaceutical Industries Ltd.: T-2001; Toyama Chemical Co. Ltd.: T-588; Phase 1:Toyama Chemical Co. Ltd.: T-817MA: former benzothiophene derivatives; Fujisawa Pharmaceutical Co. Ltd.: Tacrolimus; Berlex Biosciences: TAFI inhibitors; Eli Lilly & Co./Teva Pharmaceutical Industries Ltd.: Talampanel; GlaxoSmithKline plc: Talnetant; Loma Linda University Medical Center/Myriad Genetics Inc.: Tarenflurbil; Thromb-X NV: TB-402; R J Reynolds Tobacco Co.: TC-2429: former RJR-2429; Johns Hopkins University/Erimos Pharmaceuticals LLC: Terameprocol; NeuroSearch A/S: Tesofensine: former NS-2330; Eli Lilly & Co./TorreyPines Therapeutics Inc.: Tezampanel: former LY-293558; Kaleidos Pharma Inc.: TGF-alpha; Thuris Corp.: Thurinex; Lonza Group AG: TK-14; The Open University/EUSA Pharma Ltd.: TKP-1001; Digital Gene Technologies Inc.: TOGA technology; Telios Pharmaceuticals Inc.: TP-9201; Clinical: Merck & Co Inc.: TPA-023; Neurochem Inc.: Tramiprosate: former NC-531; Pfizer Inc.: Traxoprodil; Pharmos Corp.: Tricyclic dextrocannabinoids; Taisho Pharmaceutical Co. Ltd.: TS-011; TransTech Pharma Inc./Pfizer Inc.: TTP-4000; Universidad Complutense de Madrid: UCM-3100; University of California San Diego/Repligen Corp.: Uridine ; Vernalis plc: V-10153; Merck & Co. Inc.: V-950; Wyeth: Vabicaserin: former SCA-136; Human Genome Sciences Inc. VEGF-2 gene therapy; Oxford Biomedica plc: VEGF-targeting gene therapy; Sanofi-Aventis: Volinanserin; Vasogen Inc.: VP-025; Vertex Pharmaceuticals Inc./Taisho Pharmaceutical Co. Ltd.: VX-799; Sanofi-Aventis: Xaliprodene; Bristol-Myers Squibb Pharma Co.: XE-991; Mitsubishi Pharma Corp.: Y-931; Yungjin Pharmaceutical Co. Ltd/Korea Research Institute of Chemical Technology: YJP-60107; SK Bio-Pharmaceuticals: YKP-1358; Yuyu Inc.: YY-280; University of Gottingen: ZK-808762.

### EXAMPLE

In a cohort of older adult head trauma patients, statin users were similar to non-Statin users in terms of age (mean=75.19, sd=5.43 and mean=75.15, sd=5.52 respectively), smoking status, education, race, presence of infection, the presence of midline shift, injury severity (NISS quartile), and the likelihood of treatment in a trauma center. Statin users were more likely to have a variety of medical comorbidities including cardiovascular comorbidities, hypertension, congestive heart failure and diabetes. Statin users also took more medications than non-users. Logistic regression models which included all individuals who met inclusion criteria, regardless of whether there were injuries to other bodily areas, were tested. Without adjustment, Statin users demonstrated lower odds of dying in the hospital (O.R.=0.55, p=0.049). In multivariable analysis controlling for potential confounding variables, individuals using Statins prior to injury had significantly lower odds of in-hospital mortality (O.R.=0.49, p=0.037). As had been the case for Efron et al. (2008), an interaction between Statin use and cardiovascular comorbidities was discovered. This interaction suggested that individuals who had preexisting cardiovascular comorbidities and were on Statin therapy had much greater odds of dying before discharge, but in the multivariable logistic regression model, the interaction term was not statistically significant (O.R.=2.95, p=0.167). To understand better how the relationship between Statin use and cardiovascular comorbidities might affect likelihood of death in this population, stratified analysis was performed on two subsets of the study population. One group had no cardiovascular comorbidities, while the other was positive for at least one significant cardiovascular condition. The results of this stratified analysis suggest that, in terms of survival, individuals who had cardiovascular comorbidities did not benefit from statin use in the same manner as those without cardiovascular comorbidities possibly implicating the intact endothelium as the site of the positive effects associated with statin use.

While this description is made with reference to exemplary embodiments, it will be understood by those skilled in the art that various changes may be made and equivalents may be substituted for elements thereof without departing from the scope. In addition, many modifications may be made to adapt a particular situation or material to the teachings hereof without departing from the essential scope. Also, in the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference in their entirety. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

1. A method of treating or preventing secondary injury in a patient comprising:
administering to said patient a statin, wherein said statin is administered to said patient after experiencing a trauma that causes a brain injury, said brain injury comprising concussion, coma, infection, inflammation, a thromboembolism, or a hemorrhage, and wherein said statin is an HMG-CoA reductase inhibitor.

2. The method of claim 1, wherein said patient is on lipid lowering therapy.

3. The method of claim 1, wherein said statin is a substitute for lipid-lowering therapy.

4. (canceled)

5. A method of treating or preventing secondary injury in a patient comprising: prophylactically administering to said patient a statin, wherein said patient is at risk for a brain injury, and wherein said statin is an HMG-CoA reductase inhibitor.

6. The method of claim 1, wherein said patient is not otherwise receiving a statin in a lipid-lowering therapy.

7. The method of claim 1, wherein said statin is an HMG-CoA reductase inhibitor selected from the group consisting of lovastatin, simvastatin, fluvastatin, pravastatin, cerivastatin, rosuvastatin, atorvastatin, pitavastatin, and combinations thereof.

8. The method of claim 1, wherein said statin is administered transdermally.

9. The method of claim 1, wherein said statin prevents or mitigates endothelial dysfunction in said patient.

10. The method of claim 1, wherein said patient has a history of cardiovascular disease.

11-13. (canceled)

14. The method of claim 1, wherein said statin treats or prevents symptoms selected from the group consisting of transient confusion, loss of consciousness, becoming easily fatigued, disordered sleep, headache, vertigo, dizziness, irritability, aggression, anxiety, depression, personality changes, apathy, lack of spontaneity, amnesia, and neurologic deficits.

15-17. (canceled)

18. The method of claim 1, wherein said statin is coadministered with a second therapeutic agent.

19. The method of claim 1, wherein said statin is coadministered with a beta-blockage agent, an alpha/beta-androgenic blocking agent, an anti-inflammatory azole compound, a choline esterase inhibitor, a calcium-channel blocker, an NMDA-receptor antagonist, an antihistamine, a steroid, or a COX-2 inhibitor.

20. The method of claim 5, wherein said patient is not otherwise receiving a statin in a lipid-lowering therapy.

21. The method of claim 5 wherein said statin is an HMG-CoA reductase inhibitor selected from the group consisting of lovastatin, simvastatin, fluvastatin, pravastatin, cerivastatin, rosuvastatin, atorvastatin, pitavastatin, and combinations thereof.

22. The method of claim 5, wherein said statin is administered transdermally.

23. The method of claim 5, wherein said statin prevents or mitigates endothelial dysfunction in said patient.

24. The method of claim 5, wherein said patient has a history of cardiovascular disease.

25. The method of claim 5, wherein said statin treats or prevents symptoms selected from the group consisting of transient confusion, loss of consciousness, becoming easily fatigued, disordered sleep, headache, vertigo, dizziness, irritability, aggression, anxiety, depression, personality changes, apathy, lack of spontaneity, amnesia, and neurologic deficits.

26. The method of claim 5, wherein said statin is coadministered with a second therapeutic agent.

27. The method of claim 5, wherein said statin is coadministered with a beta-blockage agent, an alpha/beta-androgenic blocking agent, an anti-inflammatory azole compound, a choline esterase inhibitor, a calcium-channel blocker, an NMDA-receptor antagonist, an antihistamine, a steroid, or a COX-2 inhibitor.
